(19) United States
(12) Patent Application Publication
(10) Pub. No.: US 2026/0202276 A1
(43) Pub. Date: Jul. 16, 2026

(54) CONNECTION DEVICE FOR MEASURING PHYSICAL QUANTITIES OF A FLUID

(71) Applicant: Eltek S.p.A., Casale Monferrato (Alessandria) (IT)

(72) Inventors: Fabio NEBBIA, Casale Monferrato (Alessandria) (IT); Osvaldo SANDOLETTI, Casale Monferrato (Alessandria) (IT); Simone HELD, Casale Monferrato (Alessandria) (IT)

(21) Appl. No.: 19/136,730

(22) PCT Filed: Dec. 5, 2023
(86) PCT No.: PCT/IB2023/062261

§ 371 Date: Jun. 6, 2025

(30) Foreign Application Priority Data
Dec. 9, 2022 (IT) 102022000025287

(51) Int. Cl. G01L 19/00; A61M 1/36; G01L 19/06

(52) U.S. Cl. CPC G01L 19/0023; A61M 1/3641; G01L 19/0627

(57) ABSTRACT

A connection device (1) for the measurement of at least one physical quantity of a fluid comprises:-a tubular connection element (2; 2′) having a front connection portion (2b) and a rear portion (2a), the tubular connection element (2; 2′) defining a through-cavity (C₁-C₃) having a proximal opening (O₁) and a distal opening (O₂) opposite to each other, which are in the rear portion (2a) and in the front connection portion (2b) of the tubular connection element (2; 2′), respectively-a detection unit (5) capable of detecting at least one physical quantity characteristic of the fluid. The front connection portion (2b) of the tubular connection element (2; 2′) is connectable, at least at or near the distal opening (O₂) of the through-cavity (C₁-C₃), to a fluidic system (PD), in particular a device for treatment of the fluid. The detection unit (5) is mounted at the front connection portion (2b) of the tubular connection element (2; 2′), and the through-cavity (C₁-C₃) is closed at the front connection portion (2b).

### TECHNICAL FIELD

The present invention relates to a connection device for the measurement of physical quantities of a fluid. The invention has been developed with particular reference to biomedical connection devices for fluids, for medical, or biomedical, or laboratory applications, aimed at measuring a fluid pressure or other quantities.

### PRIOR ART

Connecting devices for measurement purposes are well known, particularly for use in combination with medical or biomedical equipment, where it is necessary to monitor certain characteristics of a pressurized fluid.

This is the case, for example, of certain equipment for processing bodily fluids, such as oxygenators, pumps, dialysis devices, usually used in extracorporeal circulation treatments applied to a patient, or equipment for administration or drainage of other fluids, such as a saline solution or a drug. Proper operation of equipment and devices of the indicated type typically involves constant monitoring of the pressure of the fluid entering and/or exiting the equipment, regardless of the nature of the fluid (be it a biological fluid, such as blood, or a saline solution to be infused or drained, or a drug to be infused).

In such applications, the most traditional detection solutions are based on the use of pipes equipped with a respective membrane, capable of generating a signal representative of the fluid pressure. The reliability of these detection systems presupposes a good fluidity of the liquid, and in some applications, in order to prevent possible thickening of the liquid, it may be necessary to flush the pipes frequently. This complicates the use of the equipment and leads to longer overall patient treatment times. A special case is related to blood, for which disposable pipes are normally used, which therefore do not necessarily have to be washed (although washing by means of anticoagulant substances is not ruled out). However, in the case of blood, it is of primary importance to avoid the risk of clot formation, which could give rise to serious problems (such as thrombosis or ischemia) entering a patient's circulatory system. For this reason, it may be necessary to replace the pipe during a patient's treatment, with the consequent inconveniences.

In order to overcome this drawback, connecting devices of the type of interest here have been proposed, which integrate pressure sensor means. A device of this type is known from WO 2020/194118 A, on which the preamble to claim 1 is based.

The above-mentioned document describes a biomedical connecting device for measuring physical quantities, particularly a pressure, which comprises a connection element. This tubular connection element, particularly configured in the form of a Luer-lock connector, is therefore provided with an inner through-cavity, the end or front opening of which can be connected to a biomedical device of the type indicated above. At the rear opening of the through-cavity, sensor means of the quantity of interest are provided, particularly pressure sensor means. These sensor means are mounted on a circuit support that is arranged in such a way as to close the aforementioned rear opening of the connection channel. Hence, the circuit support, particularly a small PCB (printed circuit board), is arranged orthogonally to the axis of the cavity, at the outside thereof.

A mass of gel is placed inside the through-cavity, which covers the rear opening closed by means of the circuit support bearing the sensor means. This gel, which is elastically deformable, acts as a suitable medium to transfer the quantity of interest, particularly a pressure, to the sensor means provided on the circuit support. The gel inside the cavity also has the effect of isolating and protecting the sensor means from the treated body fluid.

Although the connecting device according to WO 2020/194118 A has undoubted advantages over the more traditional technique previously indicated, it is not immune to drawbacks, related for example to the detection accuracy, the compactness and robustness of the device as a whole, the existence of risks deriving from the possible coagulation of the treated body fluid.

### SUMMARY OF THE INVENTION

In view of the above, the present invention aims to obtain a biomedical connection device for the measurement of physical quantities of an improved type compared to the known technique mentioned. In this context, one aim of the invention is to obtain one such biomedical connection device distinguished by the elimination, or at least the drastic reduction, of the risks of possible operating anomalies, in particular risks deriving from possible thickening of the treated fluid, such as a coagulation of treated blood or anomalies due to significant stagnation of the fluid inside the device.

Another aim of the invention is to obtain one such biomedical connection device distinguished a higher detection accuracy. A further aim of the invention is to obtain one such connection device distinguished by extremely compact dimensions and high structural strength.

One or more of the above aim are achieved, according to the invention, by a biomedical connection device for the measurement of physical and/or chemical quantities having the characteristics indicated in the claims. The claims form an integral part of the technical teaching provided herein in relation to the invention.

A biomedical connection device for measuring the physical quantities of a fluid according to the invention comprises a tubular connecting element, and is distinguished by the fact that its detection unit, capable of detecting at least one physical quantity of the fluid, such as a pressure, is mounted in a position corresponding to a front fluidic connection portion of the tubular connection element, i.e., a portion thereof which is intended to be connected to a fluidic system, in particular a biomedical device for the treatment of the fluid, and that the through-cavity of the tubular connection element is closed in a position corresponding to its front connection portion. The detection unit and the closing position of the through-cavity are therefore in positions closer to the area of connection to the fluidic system, represented by the front opening of the through-cavity, than to the rear opening of the same cavity.

Thanks to this configuration, the risk of thickening of the treated fluid, in particular coagulation phenomena, for example when the fluid is blood, is significantly reduced. It should be noted, in this regard, that in the device according to WO 2020/194118, a considerable part of the cavity of the connecting element requires to be filled with blood, and this favours formation of clots which, if they go into circulation, could be the cause of thrombosis or ischemia. On the other hand, in the device according to the invention, the part of the cavity occupied by the fluid may be minimal or even absent (if the detection unit is mounted right at or near the front or distal opening of the front fluidic connection portion, thus preventing or in any case drastically reducing possible blood stagnation and the related risks of clotting.

Advantageously, in various embodiments of the invention, the same detection unit is mounted to close the through-cavity, or contribute to its closure, especially a sealed closure.

In various embodiments, the detection unit is mounted inside the through-cavity, in an indented or recessed position with respect to the front or distal opening of the through-cavity. This solution makes it easier the fixing of the detection unit, which is also in a protected position. In this case, the part of the cavity occupied by the fluid is minimal, as mentioned, with a drastic reduction in fluid stagnation and the related risks of coagulation.

In various embodiments of the invention, between the detection unit and the front or distal opening of the through-cavity, a medium, such as a gel or similar mass of protective material, is placed in the cavity itself, capable of transferring at least the physical quantity from the fluid to the detection unit. In this way, if necessary, the detection unit can be additionally protected, preventing it from coming into contact with the medium, but still allowing the detection of the quantity of interest. Given that the detection unit is located in the front fluidic connection portion of the connection element (i.e., a tubular portion whose inner diameter is minimal), the aforementioned medium or gel may be in a minimal amount, i.e., in the form of a relatively thin layer, for the benefit of a better detection accuracy. In addition, such a thin layer still makes it possible to bridge the distance between the detection unit and the front or distal opening of the through cavity, thus further preventing blood pooling and clot formation.

It should be noted, in this regard, that in the devices according to WO 2020/194118, it is necessary to provide for a significant mass of the transfer medium or gel, so as to fill a significant part of the relevant cavity, in a relatively large section thereof: this can lead to errors in the transfer of the pressure to the sensitive element. In this known solution, however, it is not possible to completely fill the through-cavity of the connection element because, beyond a certain height, the mass of gel would jeopardize the accuracy of the measurement: this means that a significant part of the through-cavity of the connection element is free, and acts as a stagnation zone for the fluid being measured, favouring the formation of abnormal deposits or clots.

In various embodiments of the invention, the tubular connection element has, at the front connection portion thereof and inside the through cavity, at least one intermediate support element, to directly or indirectly support the detection unit.

In this way, at the rear or proximal end of the tubular connection element, opposite the front or distal opening, there is no need to provide a circuit support on which the sensor means has to be mounted.

Advantageously, moreover, the at least one intermediate support element can be defined integrally by the tubular element.

In various embodiments of the invention, the detection unit comprises at least one element sensitive to the at least one physical quantity, and a substrate on which the at least one sensitive element is mounted. The fact that the detection unit is located near or at the front end of the tubular connection element has the advantage that, unlike WO 2020/194118, the above-mentioned substrate can be mounted on the tubular connection element beforehand, and subsequently the at least one sensitive element can be mounted on the substrate.

In various embodiments, the substrate is sealingly constrained to the at least one intermediate support element, which ensures that the part of the through-cavity that lies downstream of the detection unit is sealed.

In various embodiments, the biomedical connection device comprises a control circuit connected in signal communication with the detection unit, the control circuit extending within the through-cavity. Preferably, the control circuit is in a position spaced apart from the detection unit, between the control circuit and the detection unit there extending first electrical conductors inside the through-cavity. However, the invention does not exclude the case of a control circuit that extends inside the through-cavity of the tubular connection element, as far as the front fluidic connection end portion, in order to directly support the detection unit and/or connect thereto.

Thanks to these characteristics, the on-board control circuitry of the biomedical connection device can be completely or at least partially housed inside the tubular connection element, to the advantage of the compactness of the device, for example compared to the one described in WO 2020/194118 (wherein the corresponding circuit is outside the cavity and orthogonal to it, with significant overall dimensions). However, it is not excluded from the scope of the invention the case in which part of the control circuitry and/or related means of electrical connection protrude from the rear end of the tubular connection element, and in this case a corresponding casing portion may be provided for the protruding part, which is associated or integrated with the tubular connection element, with such a casing portion that could be shaped to obtain at least part of an electrical connector.

In various embodiments of the invention, the biomedical connection device may include a multipolar cable, for connection of the connection device to an external system, and the control circuit comprises a circuit support bearing:
circuit components for the treatment of an electrical signal that can be acquired via the detection unit,
first terminals for connection of the first electrical conductors, and
second terminals for connection of second electrical conductors of the multipolar cable.

Such a configuration enables an easy connection of the control circuit to the detection unit, on the one hand, and to the multipolar cable, on the other hand, with this cable that can extend axially through the rear or proximal opening of the through-cavity of the tubular connection element, to the further advantage of the compactness of the device.

In other embodiments of the invention, the biomedical connection device is configured so that the circuit support has an electrical connector instead of a multipolar cable, preferably an electrical connector suitable for use in the medical or biomedical field. The electrical connector can be mounted or integrated at the rear portion of the tubular connection element, also in the form of an electrical connector of the edge connector type, integrated into the circuit support.

In this case, the device is even more compact, and only at the time of use will it have to be connected to a multipolar cable, equipped with a complementary connector, for signal connection to the external system or user device.

In various embodiments of the invention, the tubular connection element has, at least at its rear portion and inside the through-cavity, at least one of a positioning guide of the circuit support and an abutment or reference surface defining a position of maximum insertion of the circuit support inside the through-cavity. Thanks to this feature, the assembly and the precise positioning of the circuit support inside the tubular connection element is facilitated.

In various embodiments of the invention, inside the through-cavity of the tubular connection element a mass of electrically insulating fixing material, such as a resin, is arranged, to secure the control circuit and the possible multipolar connection cable in place. These characteristics contribute to increasing the structural strength of the device, also avoiding the risk of detachment between parts of the device in the event of excessive traction on the possible cable (risks that exist in the device according to WO 2020/194118).

In various preferential embodiments of the invention, the at least one sensitive element of the device comprises a pressure sensor. However, it is not excluded from the scope of the invention the detection of different physical quantities (for example a temperature) and/or the simultaneous detection of several different quantities (for example pressure and temperature) by means of one and the same detection unit equipped with several sensitive elements.

In various embodiments of the invention, the pressure sensor is a pressure sensor of the relative type, the internal cavity of which is connected in fluid communication to an ambient pressure by means of a tube, which preferably extends at least partly inside the cavity passing through, between the detection unit and the rear end portion of the tubular connection element.

The device can be made either in the form of an absolute pressure sensor (hence, sensor having a sealed internal cavity) or in the form of a relative pressure sensor, depending on the application needs.

In various embodiments of the invention, the tubular connection element is configured at least in part as a Luer-type connector, and this enables the advantageous application of the connection device in the medical or biomedical or laboratory areas. For such a case, the device may advantageously also include a fixing element, preferably a collar element, which surrounds a part of the tubular connection element at a distance therefrom, in a region thereof between the rear end and the front connection end, where the fixing element has an inner surface provided with a helical relief or thread.

The fixing element can be either formed in one piece with the tubular connection element, or configured as a separate part from the tubular connection element and rotatably mounted thereon, depending on the application requirements.

### BRIEF DESCRIPTION OF THE DRAWINGS

Further aims, characteristics and advantages of the present invention will be clear from the detailed description that follows, made with reference to the attached schematic drawings, given by way of non-exhaustive example only, wherein:

FIGS. 1 and 2 are schematic perspective views, from different angles, of a biomedical connection device in accordance with possible first embodiments;

FIG. 3 is a schematic perspective view, partially sectioned, of a biomedical connection device according to possible first embodiments;

FIG. 4 is a schematic longitudinal section of a biomedical connection device in accordance with possible first embodiments;

FIG. 5 is an exploded schematic view of a biomedical connection device in accordance with possible first embodiments;

FIG. 6 is a schematic perspective view of a component of a biomedical connection device in accordance with possible first embodiments, with some associated electrical conductors;

FIG. 7 is a schematic perspective view of the component shown in FIG. 6, from a different angle;

FIG. 8 is a partial and schematic perspective view of a front or distal end portion of a tubular connection element of a biomedical connection device in accordance with possible first embodiments;

FIG. 9 is a schematic perspective view, partially sectioned, of a front or distal end portion of a tubular connection element of a biomedical connection device in accordance with possible first embodiments, with an associated detection unit;

FIG. 10 is a partial and schematic perspective view of a front or distal end portion of a tubular connection element of a biomedical connection device in accordance with possible first embodiments, with an associated detection unit;

FIGS. 11 and 12 are schematic views, respectively in perspective and in partially sectioned perspective, of some components of a biomedical connection device in accordance with possible first embodiments, in an assembly phase;

FIGS. 13 and 14 are schematic views, respectively in partially sectioned perspective and in perspective, of the components shown in FIGS. 10-11, in an assembled condition;

FIG. 15 is a schematic perspective view of some components of a biomedical connection device in accordance with possible first embodiments, in an assembly phase;

FIG. 16 is a schematic perspective view of the components shown in FIG. 16, in an assembled condition;

FIGS. 17 and 18 are schematic longitudinal sections of a distal end portion of a biomedical connection device in accordance with possible first embodiments, respectively with and without a suitable means to transfer a physical quantity of interest;

FIG. 19 is a schematic perspective view, partially sectioned, of a biomedical connection device in accordance with possible second embodiments;

FIG. 20 is an exploded schematic view of a biomedical connection device in accordance with possible second embodiments;

FIG. 21 is a schematic perspective view, partially sectioned, of some components of a biomedical connection device in accordance with possible second embodiments, in an assembly phase;

FIG. 22 represents the detail XXIII of FIG. 21 on a larger scale;

FIG. 23 is a schematic perspective view, partially sectioned, of a biomedical connection device in accordance with possible third embodiments;

FIG. 24 is an exploded schematic view of a biomedical connection device in accordance with possible third embodiments;

FIG. 25 is a schematic perspective view of a component of a biomedical connection device in accordance with possible third embodiments, with an associated duct;

FIG. 26 is a schematic perspective view of the component shown in FIG. 26, with some electrical conductors further associated;

FIG. 27 is a schematic perspective view, partially sectioned, of some components of a connection device according to possible third embodiments, in an assembly phase;

FIG. 28 is a schematic perspective view of some components of a biomedical connection device in accordance with possible third embodiments, in an assembled condition;

FIG. 29 is a view similar to that of FIG. 28, with an additional fixing and protecting element;

FIGS. 30 and 31 are schematic longitudinal sections of a distal end portion of a biomedical connection device in accordance with possible third embodiments, respectively with and without a suitable means to transfer a physical quantity of interest;

FIG. 32 is a schematic perspective view, partially sectioned, of a biomedical connection device in accordance with possible fourth embodiments;

FIG. 33 is a schematic perspective view of some components of a biomedical connection device in accordance with possible fourth embodiments, in an assembled condition;

FIG. 34 is a similar view to FIG. 29, with an additional fixing and protecting element;

FIG. 35 is a schematic view from above of a biomedical connection device in accordance with possible fifth embodiments;

FIG. 36 is a schematic perspective view, partially sectioned, of a biomedical connection device in accordance with possible fifth embodiments;

FIGS. 37, 38 and 39 are schematic circuit diagrams, aimed at exemplifying three different possible conditioning and/or control circuits of biomedical connection devices in accordance with possible embodiments; and

FIGS. 40 and 41 are two schematic views, respectively in perspective and in section, aimed at exemplifying a possible condition of use of a biomedical connection device in accordance with possible embodiments.

### DESCRIPTION OF PREFERRED EMBODIMENTS OF THE INVENTION

The reference to “an embodiment” within this description indicates that a particular configuration, structure, or characteristic described in relation to the embodiment is included in at least one embodiment. Thus, phrases such as “in an embodiment” and the like, which may be present in different places in this description, do not necessarily refer to one and the same embodiment. In addition, particular conformations, structures or characteristics can be combined in any appropriate way in one or more embodiments, even if different from those depicted. In this description and in the attached claims, unless otherwise specified, terms such as “treated fluid”, “fluid being treated” and the like, are intended to indicate generically the fluid whose pressure or other physical quantity is to be measured by means of the device which is the subject of the invention, which can therefore be either a fluid subjected to some processing (for example oxygenation or dialysis or analysis), or a fluid given to a patient, or a fluid drained by a patient. The references used here are for convenience only and therefore do not define the scope of protection or the scope of the embodiment.

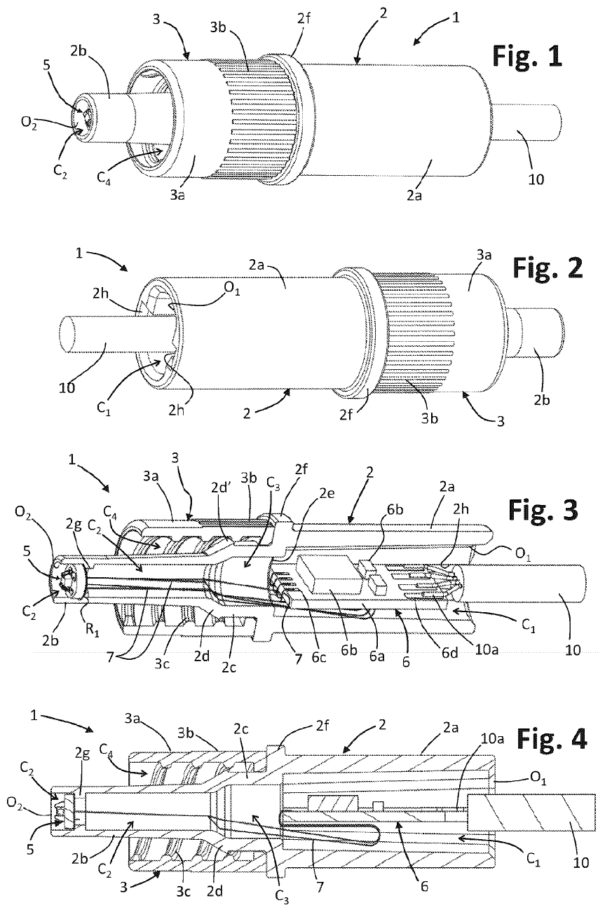

Referring initially to FIGS. 1-4, reference 1 designates a biomedical connection device for the measurement of physical quantities of a fluid, in accordance with possible embodiments of the invention. In the following, the device 1 will be described in relation to its preferential use, that is, in the medical or biomedical sector, for applications similar to those described in WO 2020/194118 A, and that the fluid being subject to detection is a body fluid, for example blood, or a fluid to be infused to, or drained from, a patient, such as a human subject or an animal.

The device 1 comprises a tubular connection element 2 or fluidic connector (hereinafter referred to as “connection element” for simplicity), preferably made of mouldable plastic material, for example a biocompatible material.

As will become clearer later, in various preferential embodiments, the connection element 2 is at least partly configured as a typical Luer connector.

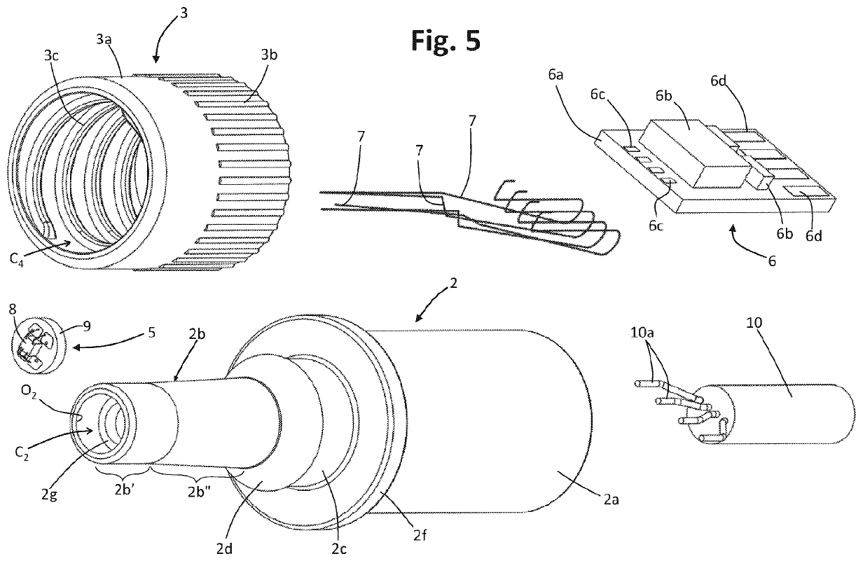

Referring also to FIG. 5, in the example illustrated, the connection element 2 has a rear or proximal portion 2a and a connecting, or front, or distal portion 2b opposite to each other and both axially extended. In the example, portions 2a and 2b extend substantially along one and the same axis, but the connection element 2 could have a proximal portion 2a and a distal portion 2b that do not extend along the same axis, for example inclined or orthogonal to each other, possibly made up of distinct portions joined together, such as a proximal portion 2a fixed or welded to a distal portion 2b. As it can be seen, both portions 2a and 2b are tubular.

The connection element 2 is shaped so as to define a through-cavity which extends between a rear or proximal end or opening O₁ and a front or distal end or opening O₂, which are in the proximal portion 2a and the distal portion 2b of the connection element 2, respectively. In the figures, C₁ and C₂ indicate the parts of the cavity that extend through the proximal portion 2a and the distal portion 2b, it being understood that the cavity itself preferentially passes through the entire connection element 2, and that its ends O₁ and O₂ are open (in FIGS. 3 and 4 only, C₃ indicates an intermediate part of the through-cavity, which is located between parts C₁ and C₂).

Preferably, the portions 2a and 2b have a substantially circular cross-section, with the maximum diameter of the rear or proximal portion 2a which is greater than the maximum diameter of the front or distal portion 2b.

In various embodiments, the portion 2b is at least partly flared or truncated-cone shape on its exterior. Preferably, but not necessarily, the portion 2b also includes a terminal stretch that is substantially cylindrical. In the example shown, the substantially cylindrical terminal section and the flared or truncated conical stretch of the distal portion 2b are designated by 2b′ and 2b″ in FIG. 5, both passed through by the cavity part C₂; preferably, at least the flared or truncated cone stretch 2b″ is substantially shaped like an analogous zone of a Luer-type connector.

In more general terms, the distal portion 2b of the tubular connection element 2 is connectable—or is configured for connection—to a user apparatus at least at or near the distal end O₂ of the through-cavity C₁-C₃. The user device is in particular a medical or biomedical device, such as a fluid treatment device, for example an oxygenator, or a pump, or a dialysis device, or a device for administering or draining a fluid, as schematized by the block PD in FIG. 17. The connection can be made, for example, by means of a flexible pipe that connects the user device PD to the biomedical connection device 1, particularly at the distal end 2b thereof. Alternatively, the connection device 1 could be directly connected to a corresponding fluidic connector of the user device PD.

Between the proximal portion 2a and the distal portion 2b, the connection element 2 may have at least one intermediate tubular portion, for example at least one shaped intermediate portion, which may include for example a generally cylindrical or generally tapered intermediate portion. In the non-limiting example depicted, as can be seen for example in FIGS. 3 and 4, there is a first intermediate portion 2c, generally cylindrical, which departs axially from the proximal portion 2a, preferably having a smaller diameter with respect thereto; portion 2c is followed by an additional intermediate portion 2d, connecting to the distal portion 2b. In this example, the intermediate portion 2d has a generally tapered or truncated conical configuration, with a greater taper angle than the distal portion 2b. In this example, the portions 2c and 2d make the intermediate part C₃ of the through-cavity (FIGS. 3 and 4).

In various embodiments, at least one intermediate portion-if present preferentially has a smaller diameter than the proximal portion, so that, within the through-cavity, a step, or at least a stop surface, is defined between the proximal and intermediate portions, the functions of which will be clarified later. In the example shown, such a step or stop surface, designated by 2e, is defined between the proximal portion 2a and the intermediate portion 2c.

Preferably, on the outside of the proximal portion 2a, on the opposite side from the end O₁ of the through-cavity C₁-C₃ of the connection element 2, an outwardly radially protruding flange is defined, designated by 2f. In this example, this flange 2f is located in an intermediate position of the connection element 2, in particular at the transition between the portions 2a and 2c of the connection element 2.

In various embodiments, the distal portion 2b has a length comprised between 7 and 13 mm (for example, referring to FIG. 5, its part 2b″ may have a length of between 7.5 and 10.5 mm, and its part 2b′, when present, a length of between 3 and 4 mm). In various embodiments, the intermediate portion 2c (downstream of flange 2f) can have a length of about 3 mm, for example 3.1 mm, while the intermediate portion 2d can have a length of about 2 mm. In various embodiments, the proximal portion 2a has a length (upstream of flange 2f) that is preferably at least equal to that of the distal portion 2b, for example between 15 and 20 mm.

In various embodiments, the biomedical device 1 comprises a fixing element, designated by 3, which is also preferably made of mouldable plastic material, for example a biocompatible material. The fixing element 3 has a circular cross-section, and is preferably shaped in the form of a ferrule or collar. The fixing element 3 (hereinafter referred to as “collar”, for simplicity) surrounds at least part of the connection element 2, at a distance therefrom, in a region of the connection element between the proximal end 2a and the distal end 2b. In this example, the collar 3 is arranged substantially coaxial to the distal portion 2b, so as to at least partially surround the portion 2b of connection element 2 (and portions 2c and 2d, if any), and define a respective annular cavity C₄. The axial dimension of the collar 3 is such that a terminal stretch of the distal portion 2b protrudes frontally with respect to the same collar 3. As can be seen, the detection unit 5 is located in the stretch of the distal portion 2b that protrudes frontally beyond the fixing element of the device 1, represented here by the collar 3. The collar 3 preferably has an outer surface, at least part of which preferably has a knurling 3b, and an inner surface, which is preferably provided with coupling or fixing means, such as a helical relief 3c. In the example, this relief acts as a thread, for screwing a corresponding male connector into the cavity C₄, for example a connector element of the user apparatus PD or a connector element placed at the end of the above-mentioned flexible pipe for connecting to the user apparatus PD, as is the case in a common Luer-type connection. The coupling or fixing means might be different from a thread, such as an element that is part of a snap coupling or a bayonet coupling.

The characteristic dimensions of the connection element 2 and the collar 3 are preferably basically standardised dimensions for Luer-type connectors. The collar 3 can have an outer diameter of between 9 and 10 mm, for example about 9.4 mm, while the cylindrical terminal stretch 2b′ of the distal portion 2b can have an outer diameter of between 3 and 4.1 mm, for example about 3.9 mm, and an inner diameter comprised between 2 and 3 mm, for example about 2.1 mm; the part of the distal portion 2b that protrudes axially with respect to the collar 3 (when the collar 3 abuts against the flange 2F) may have a length comprises between 5 and 7 mm, particularly 6.3 mm.

In various embodiments, such as that exemplified in FIGS. 1-5, the collar 3 is configured as a part separate from the connection element 2 and is mounted on it in a rotatable manner. Preferably, the collar 3 is constrained on the connection element 2 so that it can be rotated on it, but cannot be removed from it, according to a configuration known in itself in the field of ferrules for Luer-type connectors. Preferably, on the outside of the transition zone between the intermediate portions 2c and 2d of the connection element 2 there is a step or similar element (see reference 2d′ in FIG. 3 only), to hold the collar 3 when mounted on the connection element 2, so that the collar itself can move axially and rotate at the intermediate portion 2c. In the example depicted, the end 2d′ of the portion 2d that obtain the aforementioned step has an inclined stretch to ease mounting of the collar 3, and a transverse wall on the opposite side, in order to prevent the collar itself from slipping off: in this way, the collar 3 can exert a thrust on the connection element 2, during screwing the collar onto the respective connector element of the user apparatus.

As it will be seen, in possible variant embodiments, the collar can be formed in a single piece with the connection element 2. The connection device 1 can therefore be configured at least in part as a so-called two-piece Luer lock or a rotating collar Luer lock, or else as a so-called one-piece Luer lock.

Flange 2f obtains preferably a stop surface for the back face of the collar 3; for this purpose, instead of an outwardly radially projecting flange, such a stop surface could be obtained from a step or the like, defined in the tubular element 2. The connection device 1 further comprise a detection unit 5, capable of detecting at least one physical and/or chemical quantity characteristic of the fluid of interest, in particular at least one pressure.

The detection unit 5 comprises at least one element sensitive to at least one quantity of interest, indicated by 8 in FIG. 5. The sensitive element is of an electrical or electronic type and can be, for example, a pressure sensor or else a temperature sensor, such as an NTC resistor, or a combination of both. The sensitive element is preferably miniaturized, with side dimensions of less than 1 mm, for example approx. 0.7 mm.

In the following, suppose that the sensitive element 8 is a miniaturized pressure sensor, in particular comprising an integrated electronic circuit of the “die” type, made of semiconductor material, for example silicon, of a known type. This “die” circuit 8 is free of a protective casing or package, and is electrically connected to a substrate of the detection unit 5 by means of connecting microwires, which also do not have a protective coating. As it will be seen, the body of the sensitive element 8 defines a pressure-sensing membrane.

In various preferential embodiments, the detection unit 5 comprises a support of substrate of at least one sensitive element 8. Such a substrate is designated by 9 in FIG. 5. The substrate 9 is preferably formed of an electrically insulating material, e.g., a plastic material, or a ceramic material (for example alumina) or a composite material (for example FR4 or the like). Preferably, the substrate 9 has a peripheral profile corresponding to the profile of the cavity portion C₂ that crosses the distal portion 2b of the connection element 2, i.e. preferably a circular profile. The substrate 9 preferably has a thickness of between 0.5 and 1 mm, in particular about 0.75 mm, and a diameter of between 2 and 3 mm, in particular about 2.75 mm. As can be seen, the perimeter dimensions of the substrate 9 are larger than those of at the least one sensitive element 8.

According to a feature of the invention, the detection unit 5 is mounted at the distal portion 2b of the connection element 2, and the through cavity C₁-C₃ is substantially closed by the detection unit 5 at this distal portion 2b, in particular in an intermediate position of the cavity portion C₂, behind the detection unit 5. It will therefore be appreciated that the mounting position of the detection unit 5 and the closing position of the cavity C₁-C₃ are closer to the distal end or opening O₂, than to the proximal opening or end O₁, for example at a distance comprised between 0 and 10 mm from the end or opening O₂. It will also be appreciated that the mounting position of the detection unit 5 and the closing position of the cavity C₁-C₃ are located in a stretch of the cavity C₁-C₃ that has a smaller diameter (for example a diameter comprised between 2 and 3 mm), corresponding to the cavity portion C₂.

The above-mentioned mounting and closing positions are preferably within ⅓ of the overall length of the connection element 2, starting from the distal end O₂ of the cavity C 1-C₃, or within the ⅔ of length of the distal portion 2b, starting from the same distal end O₂.

In preferential embodiments, it is the same detection unit 5 that is mounted in such a way as to close the through-cavity C₁-C₃ within the distal portion 2b, or contribute to the closure of said cavity.

Preferably, the detection unit 5 is sealingly mounted inside the through-cavity, in particularly in its portion C₂ corresponding to the distal portion 2b. The detection unit 5 is preferably mounted in a recessed position with respect to the distal end O₂ of the cavity C₁-C₃. Preferably, the unit 5 is located in a recessed position that is very close to the end O₂ of the distal portion 2b; the distance between the upper surface of substrate 9 and the end O₂ can be comprised between 1 and 1.5 mm, in particular about 1.1 mm, but it can be greater, for example up to 7 mm.

To this end, in various embodiments, the connection element 2 has, at the distal portion 2b thereof and within the relevant cavity portion C₂, at least one support or rest element, for the fixing and/or support of the detection unit 5.

An example of such a support element is indicated by 2g in the figures, and is preferably defined by the body of the same connection element 2, within the cavity portion C₂, near its distal end O₂. Preferably, as can be seen in particular from FIGS. 8 and 9, the at least one support element 2g comprises a flange formation which is radially protruding towards the inside of the cavity portion C₂, starting from the inner surface of the distal portion 2b.

The support element 2g therefore has preferably an annular profile, which is substantially circular, and defines a support surface for the back or rear surface of the substrate 9 of the detection unit 5; on the other hand, to the front or forward surface of the substrate 9, the at least one sensitive element 8 is associated. The support element 2g can protrude from the inner surface of the distal portion 2b by less than 0.5 mm, in particular about 0.4 mm.

Preferably, the support element 2g is at a distance from the distal end O₂ of the through-cavity such that the sensitive element 8 is in a recessed position with respect to said distal end, as can be seen for example in FIGS. 8 and 9. In possible variants, the support element could be at the distal end O₂ of the through-cavity, for example for use of the device in combination with non-chemically aggressive and/or non-electrically conductive media, in particular with a connection of the sensitive element 8 to the substrate 9 by SMD technique.

In various preferential embodiments, the substrate 9 is sealingly constrained on the at least one support element 2g. A sealed fastening can be achieved, for example, by placing a resin or other bonding material on the top surface of the support element 2g, and then placing the substrate 9 on it and/or placing the resin or glue on the substrate 9 and then placing it on the support 2g, so that the detection unit contributes, with the support element 2g and the bonding material, to the closure of the through-cavity C₁-C₃. Such a bonding layer of the substrate 8 is indicated for example by R₁ in FIG. 17. The substrate 9 does not necessarily have to rest and be fixed directly on the support element 2g, since an intermediate element may be provided therebetween between, which in turn is fixed to substrate 9, on the one hand, and to the support element 2g, on the other hand.

In various embodiments, the biomedical connection device 1 comprises a conditioning and/or control circuit (hereinafter referred to as “control circuit”, for simplicity's sake), which is connected in signal communication with the detection unit 5. Preferably, the control circuit is integrated into the biomedical device 1, or extends at least partly within its through-cavity C₁-C₃. Very preferably, the control circuit extends axially into the cavity C₁-C₃ and can be in a position spaced apart from the detection unit 5: in this case, the corresponding electrical connecting conductors extend between the control circuit and the detection unit.

Such a control circuit is indicated by 6 in the figures. As can be seen in particular in FIGS. 3 and 4, the circuit 6 is mounted inside the cavity portion C₁ corresponding to the proximal portion 2a of the connection element 2, that is, the portion of cavity C₁-C₃ with the largest diameter.

As shown in FIG. 5, in various embodiments, the circuit 6 comprises a circuit support 6a, preferably formed of electrically insulating material, such as a plastic material, or a ceramic material (for example alumina) or a composite material (for example FR4 or the like). The support 6a can advantageously be a suitably sized PCB.

Circuit components (electrical and/or electronic) are mounted on the circuit support 6a for the processing of an electrical signal-representative of the quantity of interest-which can be acquired by means of the detection unit 5, in particular by means of its sensitive element 8.

As mentioned, preferably, the circuit 6 is in a position at a distance from the detection unit 5, and the two parts are connected to each other by means of electrical conductors, such as the conductors designated by 7 in the figures, which are preferably flexible conductors with surface insulation. In order to allow the electrical connection between the detection unit 5 and the circuit 6, the latter provides, on the support 6a, corresponding first terminals or pads 6c, for the connection of the conductors 7.

Given that the circuit 6 must also be connected to an external system (exemplified by block CS in FIG. 17, which can be a control system of the same apparatus PD) for the processing of the detected signals, and possibly for the power supply, the connection device 1 is preferably also provided with means for electrical connection and/or communication, such as a multipolar cable, designated by 10, and on the support 6a there are provided second terminals or pads 6d, for the connection of the conductors 10a of the cable 10. The electrical connection between the aforementioned terminals and conductors can be made by soldering or an electrically conductive paste.

In variants not shown, the device 1 may be equipped with a stand-alone power source, such as a battery, and/or its on-board circuitry may be configured for an inductive power supply. The control circuitry of the device 1 could also be configured to perform a wireless data communication with the external system CS.

The connection of the conductors 7 to the sensitive element 8 of the detection unit 5 can be carried out by providing suitable connection elements on the substrate 9 of the same unit.

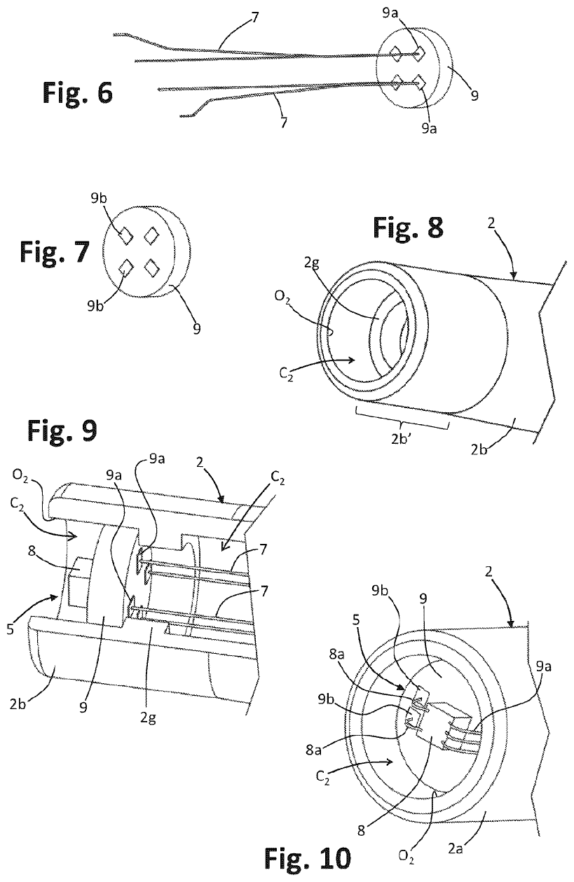

Referring for example to FIGS. 6-8, in various embodiments the substrate 9 has, on its back face, first connecting terminals or pads 9a, formed of electrically conductive material, to which the end of the conductors 7 opposite the circuit 6 can be soldered or associated via an electrically conductive paste. On the other hand, on the front face of the substrate 9 there are similar second connecting terminals or pads 9b, which are also made of electrically conductive material.

Each terminal 9a is electrically connected to a respective terminal 9b in a known manner. For example, the terminals 9a and 9b may be in staggered or axially aligned positions, and the substrate 9 may have through-holes in positions corresponding to said terminals, wherein the aforementioned holes are filled with an electrically conductive material (or have surfaces covered with an electrically conductive material) that ensures electrical continuity between the terminals 9a and 9b. Such a case is exemplified in FIG. 17, where 9c designates an electrically conductive material that fills the relevant through-holes of the support 8 and extends between the terminals 9a and 9b.

The connection of the sensitive element 8 to the terminals or pads 9b may also be carried out in accordance with known technique. For example, in the case where the sensitive element 8 is—as in the preferential case shown—of the type comprising an integrated electronic circuit of the “die” type made of semiconductor material (for example silicon), its electrical connection to the terminals 9b can be obtained by wire bonding, i.e. by means of microwires made of electrically conductive material (preferably a noble metal) extending between the terminals 9b and corresponding pads (not highlighted) of the sensitive element 8. Some of these connecting microwires are indicated with 8a in FIGS. 10 and 17 (in FIG. 9 the aforementioned microwires are not represented for the sake of greater clarity of representation).

As mentioned, preferably, the detection unit 5 is in a recessed position with respect to the distal end O₂ of the cavity C₁-C₃. In this way, the sensitive element 8 and its microwires 8a are in a protected position. This protection is further enhanced in those embodiments in which a mass of a protective material, such as a gel, is placed in the volume comprised between the distal end O₂ and the upper surface of the substrate 9.

From FIG. 17 it can also be noticed a possible structure of a miniaturized pressure sensor that can be used to implement the invention. The sensor 8 has a body that comprises a “die” made of semiconductor material, in which a cavity 8b is defined. In this way, the upper part of the “die” which is in a position corresponding to cavity 8b obtains a membrane portion 8c, which can be deformed by the pressure of the fluid of interest. As per the known technique, in this membrane portion 8c suitable means are implemented, to detect the degree of deformation of the membrane (for example a plurality of resistances in a Wheatstone bridge configuration), with an output signal that is representative of a pressure value.

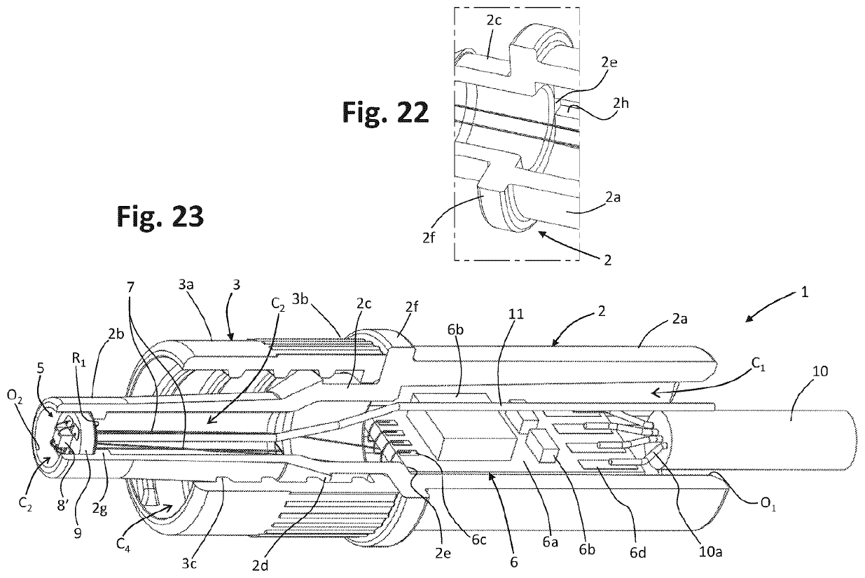

The positioning of the control circuit 6, or of its support 6a, within the proximal portion 2a, or the corresponding cavity portion C₁, is carried out by inserting the same support through the end O₁ of the through-cavity C₁-C₃. In order to ease and make this insertion precise, it is preferable that the connection element 2 has, at least at the proximal portion 2a thereof and inside the through-cavity, at least one positioning seat or guide for the support 6a. In various preferential embodiments, this positioning guide comprises two guides or seats defined in diametrically opposite parts of the cavity portion C₁, which extend starting from the corresponding end O₁. The guides advantageously comprise grooves, such as those designated by 2h in FIGS. 2, 3, 12, 14 (see also FIGS. 21 and 22 for reference), which extend axially within the cavity portion C₁.

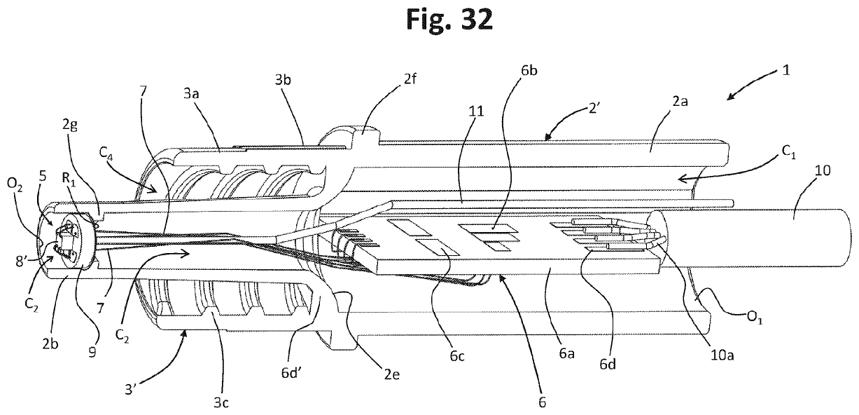

Preferably, the connection element 2 has, at least at the proximal portion 2a thereof and inside the through-cavity, at least one abutment or stop surface, defining a position of maximum insertion of the circuit support 6a. Such a stop surface can be represented by the same surface already designated by 2e, as can be seen for example from FIGS. 3 and 13 (see also FIGS. 23 and 32 for reference).

As can be imagined, the opposite longitudinal edges of the circuit support 6a can be inserted into the aforementioned grooves 2h, and the support itself can be slid towards the inside of the cavity portion C₁, until the front edge thereof abuts against the stop surface 2e.

A possible assembly of the described connection device is as follows.

The distal ends of the conductors 7 are soldered to the corresponding terminals 9a on the back of the substrate 9 of the detection unit 5, for example as shown in FIG. 6; the soldered joints can be protected via deposition of an epoxy resin. Subsequently, a layer of fixing and sealing resin is placed on the surface of the support element 2g (FIG. 8) and/or the substrate 9; as mentioned, such a layer is designated by R₁ in FIG. 17.

The ensemble of conductors 7—substrate 9 is then inserted through the distal end O₂ of the through-cavity of the connection element 2 until the back of the substrate 9 rests on the support element 2g and/or the resin layer R₁. Following polymerization of the resin R₁, the substrate 9 is sealingly fixed, in particular at the support element 2g (that is, in the position shown in FIGS. 9 and 10, although these figures already show the sensitive element 8). Note that the conductors 7 are longer than the axial cavity of the connection element 2, so that a portion of the conductors including their proximal ends protrudes outside the through-cavity, beyond the proximal end O₁ of the cavity.

The sensitive element 8 is then placed on the front of the substrate 9 and constrained thereto, for example by gluing or another well-known “die attach” technique. This is followed by the electrical connection of the sensitive element 8 to the corresponding terminals 9b on the front of the substrate 9; as mentioned, in the non-limiting example considered, this is carried out by wire bonding, using microwires 8a as in FIG. 10 or 17.

The proximal ends of the conductors 7 are then soldered to the corresponding terminals 6c present on the circuit support 6a, and the conductors 10a of the multipolar cable 10 are soldered to the corresponding terminals 6d present on the same support 6a (see FIG. 3).

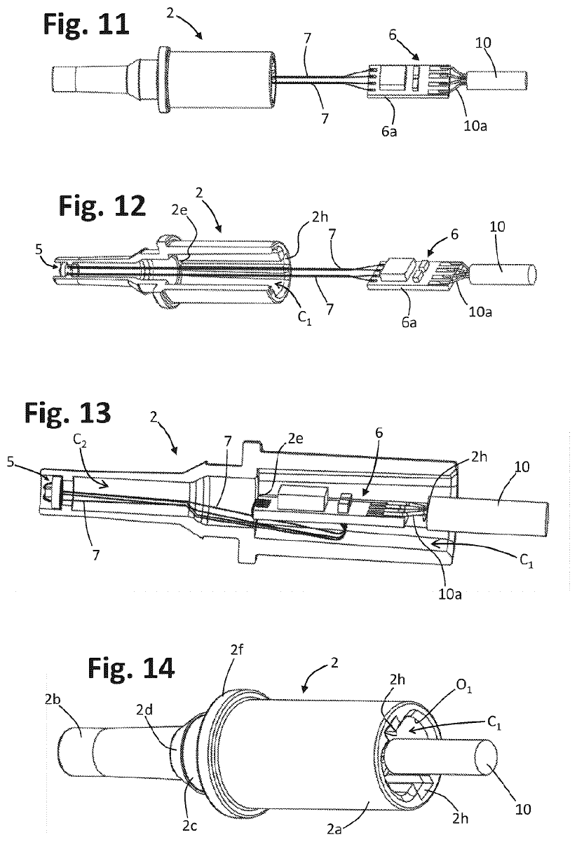

Also in this case, the soldered joints can be protected by deposition of an epoxy resin. Such a step in the assembly process is shown in FIGS. 11 and 12.

Preferably, a glue is deposited on at least part of the guides 2h (FIGS. 2-3) provided within the cavity portion C₁, for example on their bottom (in addition to or alternatively, the aforementioned glue can be placed on at least one part of one or both the longitudinal edges of the support 6a).

The bottom of said guides 2h is defined in particular by the stop surface previously designated by 2e (see FIGS. 12-13). The longitudinal edges of the circuit support 6a bearing the corresponding components are then inserted into the corresponding guides 2h and the same support 6a is inserted into the cavity portion C₁, until its front edge abuts against the stop surface 2e, preferably provided with the aforementioned glue, as shown in FIGS. 13 and 14. The subsequent curing of this glue fixes the circuit support 6a in place, and this is preferable in view of the subsequent introduction of a mass of sealing resin, as explained below. During the phase of insertion of the support 6a, the conductors 7, preferably flexible, adapt their configuration within the through-cavity, for example as shown in FIG. 13. Following insertion, the cable 10 protrudes from the proximal end O₁ of the through-cavity C₁-C₃, as shown for example in FIG. 14. An end portion of the cable 10 remains preferably within the cavity portion C₁.

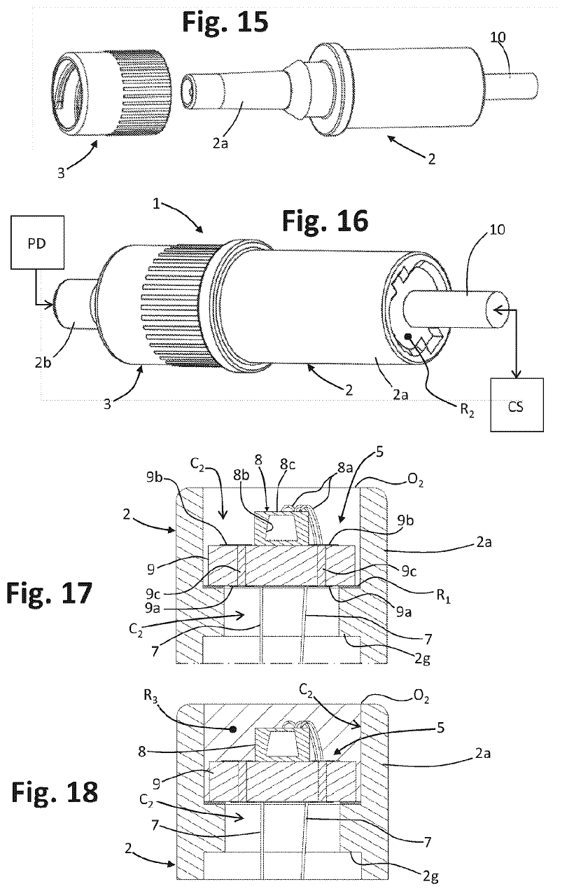

The collar 3 is mounted on the connection element 2, as shown in FIG. 15, and inside the through-cavity of the same element 2, a mass of electrically insulating fixing material is introduced, through the proximal end O₁, for example a resin, designated by R₂ in FIG. 16, which, following polymerization, seals at least the circuit 6 in place and also fix the cable 10 relative to the connection element 2. Of course, the sequence of the last two indicated steps (mounting of the collar 3 and application of the resin R₂) can be reversed.

Preferably, a means for transferring a physical quantity from the fluid to be detected to the detection unit 5, or to the sensitive element 8 thereof, is arranged between the detection unit 5 and the distal end O₂ of the through-cavity C₁-C₃. In the case of a sensitive element 8 designed to detect pressure, this means is an elastically deformable, and preferably electrically insulating, means. The aforementioned means may be, for example, a mass of gel or similar protective material, for example a biocompatible gel. Such a means or mass—designated by R₃ in FIG. 18—therefore protects the sensitive element 8 (and the corresponding microwires 8a, when provided) from direct contact with the fluid, while allowing transfer of pressure to the sensitive element 8, in particular to the membrane portion 8c thereof. The protective mass R₃ is therefore susceptible to be in contact with the fluid, and to separate the fluid itself from the detection unit.

It will be appreciated that, unlike the devices according to WO2020/194118, the proximal end portion 2a of the connection element 2 can be configured according to various shapes, even different from the one exemplified, in order to obtain or integrate a housing casing for the circuit support 6a. In devices according to WO2020/194118, the proximal end of the tubular connection element must instead be configured to adhere tightly with an external circuit support, in order to be able to be filled with a gel without leakage, and with the additional complication that the seal must be guaranteed even when the gel is subjected to thrust due to fluid pressure. The known solution therefore requires a large circuit support, such as to include the external profile of the proximal end of the tubular connection element:
by contrast, the solution according to the invention allows the possible use of a smaller circuit support, with obvious savings in terms of production. The device known from WO2020/194118 is also delicate and this circumstance limits its possible use, necessarily having to provide at least one additional protective casing for the circuit support. This additional casing must be mechanically fixed to the tubular connection element, in such a way as not to transfer to the circuit support mechanical stresses to which the tubular connection element is subjected during connection/detachment from the respective connector of the user apparatus: this determines obvious structural complications, which typically involve larger overall dimensions and higher production costs, absent instead in the solution according to the invention.

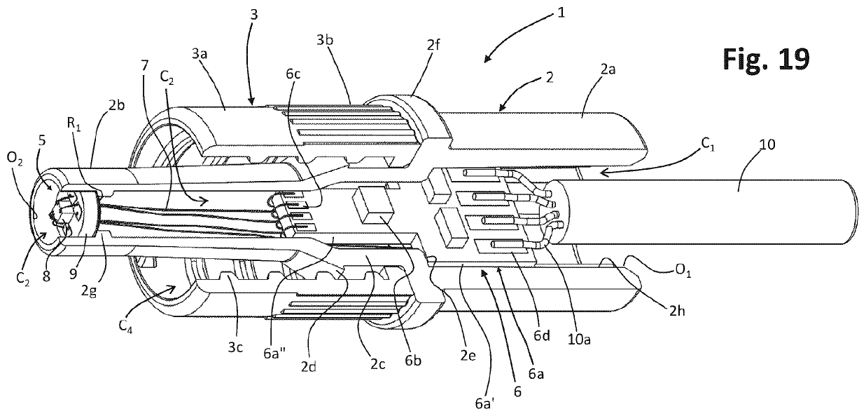

The biomedical connection devices of FIGS. 19-22 are similar in design to the biomedical devices of FIGS. 1-18, but with a different configuration of the circuit support.

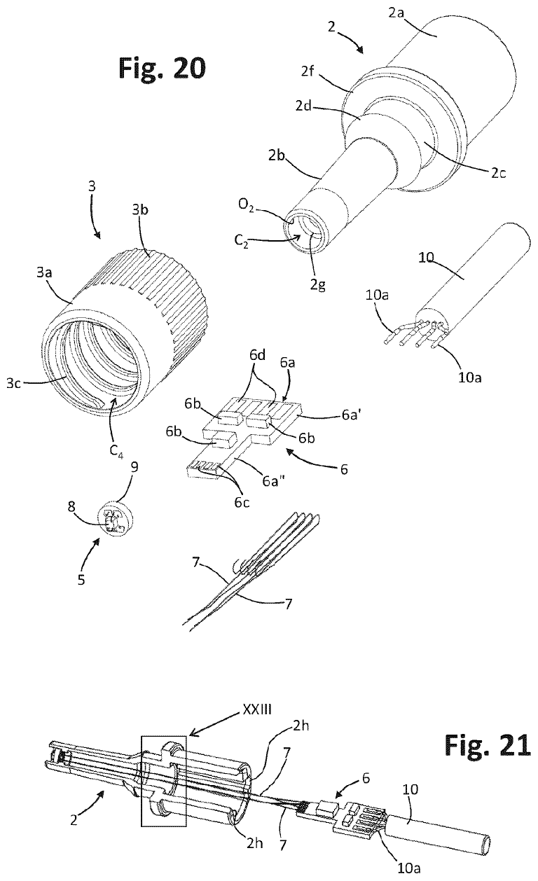

As can be seen in particular in FIGS. 19 and 20, in this case the body of the circuit support 6a has a rear part 6a′, to which there are preferably associated the terminals or pads 6d for connection of the conductors 10a of the cable 10, and a front part 6a″, to which there are preferably associated the terminals or pads 6c for connection of the conductors 7 belonging to the detection unit 5. The front part 6a″ has a reduced width compared to the rear part and with respect to the distance in the diametral direction between the guides 2h, or with respect to the corresponding bottom surfaces 2e (see the detail in FIG. 22). The rear part 6a′ of the support 6a is suitable for insertion in the proximal portion 2a and in the corresponding guides 2h, while the front part 6a″ is suitable for insertion in at least part of the intermediate portion 2c, and possibly of the intermediate portion 2d, and possibly of the distal portion 2b.

In this way, the front part 6a″ of the circuit support 6a may be located closer to the distal end 2b of the connection element 2, for example within the intermediate portions 2c and 2d, and possibly at least also partly within the distal portion 2b. Such a solution may, for example, be useful to provide a larger surface area on the circuit support 6a for the arrangement of the circuit components 6 and/or to reduce the length of the conductors 7 and/or to allow—with the same surface area of the support 6a compared to the case of FIGS. 1-18—a greater insertion of the multipolar cable 10 within the cavity portion C₁, for the purpose of improving its anchoring to the connecting element 2.

For the remainder, the assembly of the biomedical connection device 1 of FIGS. 19-22 may be similar to that described above.

A configuration of the circuit support as shown in FIGS. 19-22 can also be used in the other embodiments described here.

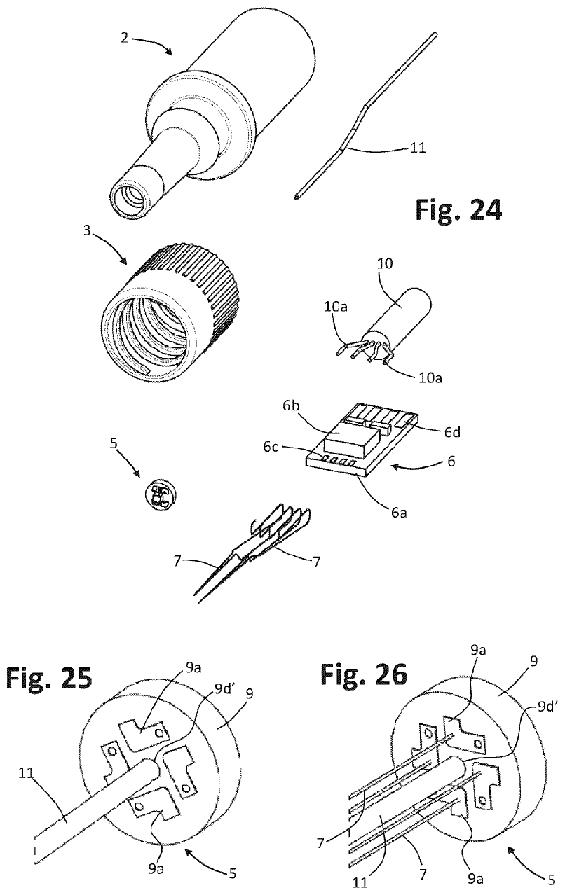

The biomedical connection devices of FIGS. 23-31 are similar in design to the devices of FIGS. 1-18 and 19-22, but are distinguished by a different type of sensitive element.

Also in the case exemplified in FIGS. 23-31, the sensitive element—designated by 8′—is a pressure sensor, but of the so-called “relative” type, that is, of the type that requires a reference pressure (typically the ambient pressure) for the purpose of detecting the fluid pressure.

In this regard, it should be noted that in the example in FIGS. 17-18, the pressure sensor 8 is of the so-called “absolute” type, whose internal cavity 8b is sealed with respect to the environment, thus determining a predefined reference pressure into the cavity 8b.

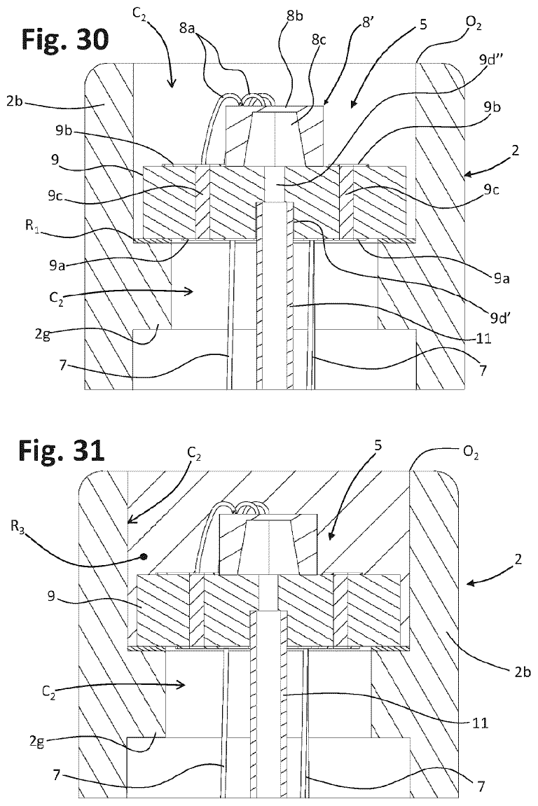

As can be seen in FIG. 30, the sensor 8′ also defines an internal cavity 8c, which is connected in fluid communication to the ambient pressure by means of a small tube, designated by 11 also in FIG. 31. As can be seen in FIG. 23, the tube 11 extends inside the through-cavity of the connection element 2, between the detection unit 5 and the proximal end portion 2a.

Referring again to FIG. 30 as well as to FIGS. 25 and 26, in this case the substrate 9 of the detection unit 5 is provided with a through-hole, preferably in a central position.

The hole has a lower portion 9d′, which opens at the back of the substrate 9, to which the distal end of the tube 11 is coupled, as shown in FIG. 30. From the same figure it can be seen that the upper portion 9d″ of the hole opens instead at in a position corresponding with the cavity 8c of the sensor 8′. Preferably, the two portions 9d′ and 9″ of the hole have different diameters, the first having a larger diameter to receive the end of the tube 11.

For assembly purposes, in this case, the distal end portion of the tube 11 is inserted into the lower portion 9d′ of the hole of the substrate 9, and possibly fixed in place by means of a glue (obviously taking care not to obstruct the tube itself). Next, the distal ends of the conductors 7 are soldered to the terminals 9a on the back of the substrate 9, and then the assembling proceeds in a manner similar to what has been described above.

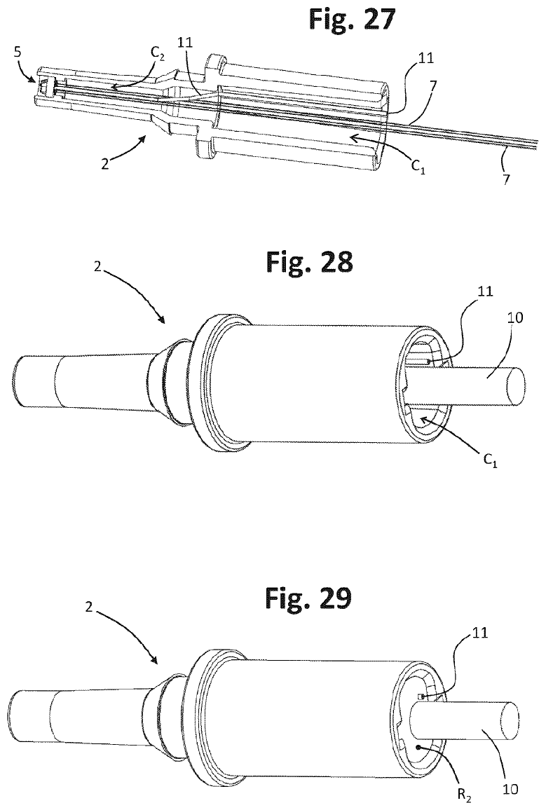

The ensemble of conductors 7—tube 11—substrate 9 is inserted through the distal end O₂ of the through-cavity of the connection element 2, up to the abutment of the back of the substrate 9 against the support element 2g and/or the resin layer R₁, with the proximal end of the tube 11 being substantially at the end O₁ of the through-cavity of the connection element 2, or slightly protruding therefrom, as can be seen in FIGS. 27-28.

After connection of the conductors 7 to the substrate 6a of the circuit 6 and after insertion of the circuit 6 (FIG. 28) into the connection element 2, as explained above, the mass of fastening material R₂ is introduced into the through-cavity of the connection element 2 through the proximal end O₁, as shown in FIG. 29, which, after polymerization, seals at least the circuit 6 and the tube 11 in place, and also fixes the cable 10 relative to the connection element 2. Of course, the mass of the fixing material R₂ is in any case such as to leave the proximal end of the tube 11 exposed, for the necessary sampling of the reference ambient pressure, as shown in FIG. 29.

Also in this case, as can be seen in FIG. 31, between the detection unit 5 and the distal end O₂ of the through-cavity, the means R₃ is preferably arranged, suitable for protecting the sensitive element 8′ with the corresponding microwires 8a, and suitable for transferring the pressure from the fluid subject to detection to the sensitive element represented by the relative pressure sensor 8′.

A design of the detection unit 5 with a corresponding absolute pressure sensor and the corresponding tube 11, as shown in FIGS. 23-31, can also be used in the other embodiments described here.

The biomedical connection devices shown in FIGS. 32-34 are substantially similar in design to the devices of FIGS. 23-31, but are distinguished by a different design of the connection element. In particular, in this case, the collar, designated here by 3′, is formed in a single piece with the tubular connection element or fluidic connector, here designated by with 2′, obtaining, for example, a Luer type connector with a one-piece coupling or “one piece Luer lock”.

The characteristic in question can be seen, for example, in FIG. 32, which shows how the collar 3′, or its parts indicated by 3a-3c, are in this case defined in a single piece with the connection element 2′. From the same figure it can be seen that, in this example, between the proximal portion 2a and distal portion 2b of the connection element 2′ there is a single intermediate portion 6d′ having a generally tapered shape, which forms a transition between the cavity portions C₁ and C₂, and which is in any case suitable for obtaining the stop surface 2e that determines the end-of-stoke to the insertion of the circuit support 6a within the cavity portion C₁.

In the example, the detection unit 5 includes a relative pressure sensor 8′, to which a corresponding tube 11 is associated, but the one-piece construction of parts 2′ and 3′ can of course be also used in embodiments that uses an absolute pressure sensor 8. More generally, a design of the connection element 2′integrating the corresponding collar 3′in a single piece, as shown in FIGS. 32-36, can also be used in the other embodiments described here.

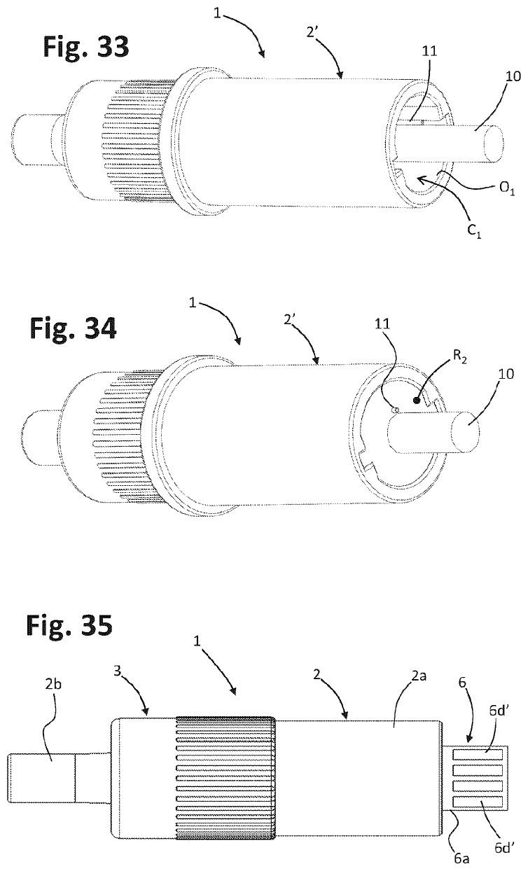

The assembly of device 1 is similar to the one previously described with reference to FIGS. 23-31, as can also be seen from FIGS. 33-34 (which illustrate assembly phases substantially corresponding to those of FIGS. 27-29): of course, in this case, it will not be necessary to assemble the collar 3′ on the connection element 2′, as it is already integrated in it.

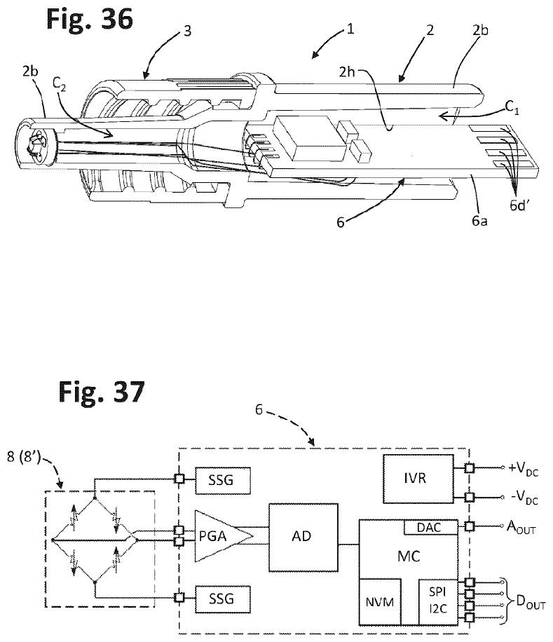

The biomedical connection devices referred to in FIGS. 35 and 36 are substantially similar in construction to the devices in the previous figures, but without the multipolar cable 10, that is, they are distinguished by the presence of an electrical connector for the connection of a complementary electrical connector, for example provided at the end of a connection cable towards the system CS of FIG. 17.

In embodiments of this type, the support 6a of the circuit 6 may have a longer length in the axial direction than in the previous cases, so that one of terminal stretch thereof protrudes outwards (for example by about 3.5-4.5 mm) of the cavity portion C₁, through the end O₁, as shown in FIGS. 35-36. On this protruding portion of the support 6a, terminals or pads 6d′ are provided near the edge of the same support, which is advantageously configured as a PCB. In this way, as can be imagined, the protruding part of the support 6a equipped with the terminals 6d substantially obtains the male part of an electrical connector system, for example of the edge connector type. In this case, the external system CS or the multipolar wiring extending towards the external system CS will be equipped with a corresponding female electrical connector.

Even in this type of embodiment, the mass of electrically insulating fixing material previously designated by R₂ is introduced into the through-cavity of the connection element 2, through the proximal end O₁ (see FIGS. 16, 29 and 34 for reference), which seals the support 6a in place after polymerization.

The assembly of device 1 is similar to that described above with reference to the other embodiments: of course, in this case, it will not be necessary to connect the terminals of a multipolar cable directly to the circuit 6. A design of the support 6a which includes a corresponding electrical connector, as shown in FIGS. 35-36, can also be used in the other embodiments described here.

The circuit 6 can be configured to perform an active compensation of the signals acquired through the relevant sensor means. In various embodiments, the circuit 6 comprises all the circuitry required for amplification, compensation, and conversion of the aforementioned signals.

Referring for example to FIG. 37, IVR designates a voltage regulator of the sensor system, which is supplied with a low DC voltage (for example 5 V DC), while with SSG are generically designated the components used to apply the supply voltage to the sensitive element 8 or 8′.

PGA designates an analog signal amplifier, which has the purpose of adapting the level of the differential signal coming from the sensitive element 8 or 8′, to obtain an amplitude suitable for being processed by the next block AD, representing an analog/digital converter, capable of converting the analog signal from the sensitive element 8 or 8′ into a numerical form, for further processing.

MC designates a microcontroller that manages the signal conditioning, that is, carries out the processing necessary to convert the digital signal provided by the converter AD into the desired information to be produced at the output. Preferably, the parameters required for such processing (together with the gain of the amplifier PGA and the resolution required for the converter AD) are stored, during the system calibration phase, in a non-volatile memory NVM (in this way, the microcontroller MC may be able to self-configure each time the system is turned on).

The result of the processing is finally made available on the digital outputs D_{OUT}, for example via the interfaces designated by I2C and SPI, or converted by the digital/analog converter designated by DAC to an analog voltage, made available on a dedicated terminal A_{OUT}.

Alternatively, the on-board circuitry of the device 1 can be configured to perform a passive compensation: in this case, the signal coming from the sensitive element 8 or 8′ is modified by interposing appropriate resistors in series and/or in parallel, the values of which are set during the calibration phase, for example by means of appropriate cutting performed with laser during the production cycle.

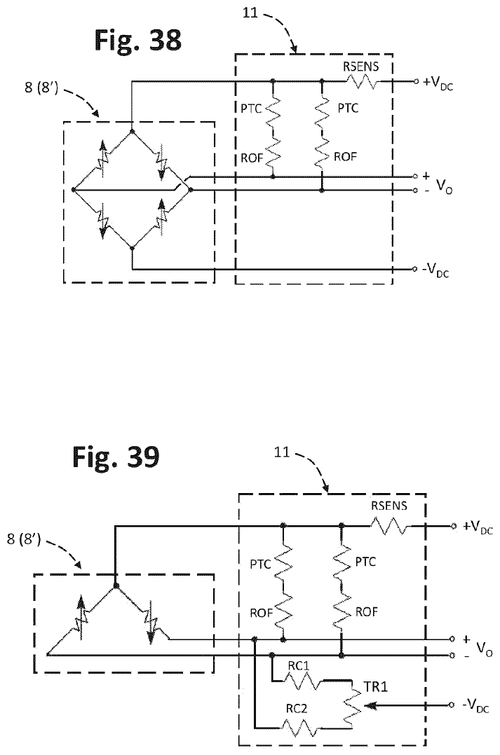

Referring for example to FIG. 38, with PTC temperature-sensitive resistors are designated, which allow to compensate for possible changes in the signal due to a change in the temperature of the environment in which the system is located during operation. ROF designates balancing resistors, in order to balance the output of the sensitive element 8 or 8′ to have a zero voltage V₀, for example when the applied pressure of the fluid is zero. With RSENS, on the other hand, a calibratable resistor is indicated (for example a resistor that is calibratable or trimmerable by means of laser cutting or directly weldable): by acting on this element it is possible to vary sensitivity from the sensitive element 8 or 8′, or the ratio between the variation of the output voltage V₀ with respect to the variation of the physical quantity, such as an applied pressure.

In other possible embodiments, the sensitive element 8 or 8′ may include only two resistors, in a half-bridge configuration. Such a case is exemplified in FIG. 39, which adopts a circuit layout partially similar to that of FIG. 38, in that it also employs the temperature-sensitive resistors PTC, the balancing resistors ROF, and the calibratable resistor RSENS.

The sensitive element 8 or 8′ of FIG. 39 is connected to the control circuit 11 by means of only three electrical wires, and in this case the circuit itself provides two additional resistors, designated by RC1 and RC2, to close the output signals from the element 8 or 8′ on the negative. These additional resistors RC1 and RC2 can be of a fixed value, typically a value comparable to that of the impedance of the sensitive element (but if necessary they can have values even considerably higher, if a particular common mode is required at the output). If necessary, the circuit arrangement can in this case also include a variable resistor or trimmer, designated by TR1, adjustable during calibration, which could obviously be replaced by calibratable resistors, for example an appropriate TR1 resistor that can be calibrated, for example by laser cutting, and/or the same additional resistors RC1 and RC2 could be of a calibratable type, for example via laser cutting.

The configuration shown in FIG. 39 is advantageous in that it allows for the use of a half-bridge sensitive element 8 or 8′, which typically has smaller dimensions than a full-bridge sensitive element of the type shown in FIGS. 37 and 38, and enables to reduce the number of electrical connections between the element 8 or 8′ and the circuit 11 (three connection wires 7 instead of four), also facilitating the bonding operations.

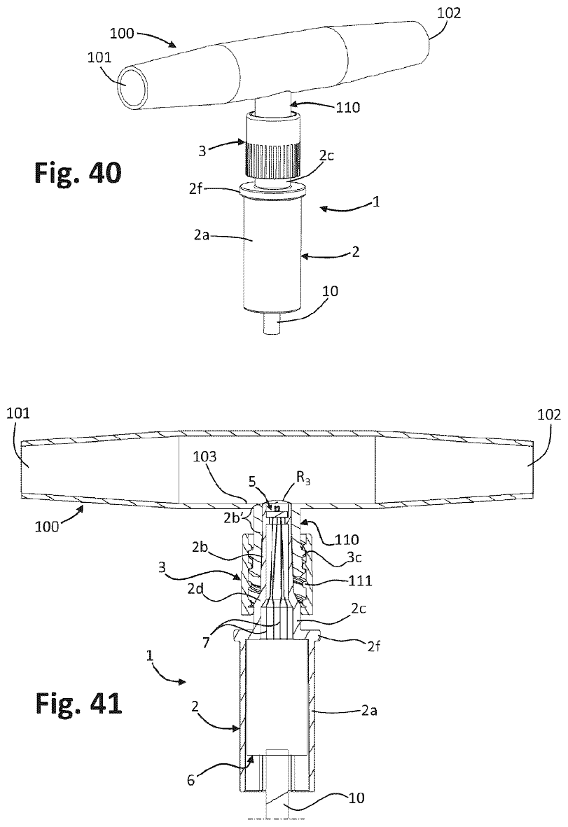

FIGS. 40 and 41 schematically show a possible condition of the use of a device 1 according to the invention. In the exemplified case illustrated, the biomedical connection device 1 is coupled to a generic tee fitting, designated as a whole by 100, having an inlet 101 and an outlet 102 for connection in a fluidic circuit, belonging for example to a dialysis machine. The fitting 100 includes an axially extended intermediate tubular connection 110, which in this example is configured at least in part as a female Luer connector. For this purpose, as can be seen in FIG. 41, a thread 111 is defined on the outer surface of the fitting, suitable for engaging with the internal thread 3c (see FIG. 2) of the collar 3 of the device 1. The inner surface of the tubular connection 110 may be at least partially truncated-cone shaped, with a taper substantially complementary to the homologous truncated-cone stretch (2b″, FIG. 5) of the front connection portion 2b of the device 1.

As indicated above, the front connecting portion of the biomedical device 1 may have a substantially cylindrical terminal stretch 2b′ (see also FIG. 5), and preferably also the inner surface of the connection 110 has a corresponding stretch with a cylindrical surface, in order to couple with the stretch 2b'. These cylindrical stretches facilitate the correct final reciprocal positioning between the device 1 and the tubular connection 110. As can be seen in FIG. 41, the portion 2b of the device 1 is inserted into the connection 110, and the collar 3 of the same device is tightened, to lock the two parts in position. Preferably, in the final locked condition, the distal end of the portion 2b is near or substantially flush with the surface 103 defining the inner duct of the fitting 100, i.e., the duct that extends between the inlet 101 and the outlet 102. In this condition, the detection unit 5 is in a position slightly recessed with respect to the surface 103, in order to be able to detect with precision the pressure of the fluid which passes through the fitting 100. The absence of possible fluid stagnation can be avoided thanks to the presence of the mass of gel R₃, as previously explained.

From the given description the characteristics of the present invention are clear, as also clear are its advantages.

It is clear that numerous variations are possible for the person skilled in the art to the device described as an example, without however departing from the scope of the invention as defined by the claims that follow.

As indicated, the at least one physical quantity of interest may be different from the pressure, such as the temperature of the fluid and/or of the environment in which the device operates, in which case on the substrate 9 and/or the support 6a of the control circuit 6 there will be mounted a corresponding sensor means, of a known type. The detection of the ambient temperature can be useful in order to compensate the measurement carried out by the sensitive element (for example to compensate possible thermal drifts of the electronic components, both at the level of the measuring bridge present in a pressure sensor, and of components mounted on the internal circuit).

The proximal end portion of the tubular connection element and/or a portion of the casing associated with or integrated with said proximal end portion may be shaped to obtain at least part of an electrical connector. For example, the proximal end portion may have a tubular portion, preferably shaped in such a way as to create coupling and/or hooking and/or positioning elements, complementary to a respective connector of the external system CS. Such elements may be advantageously configured in order to avoid incorrect connection, and may include for this purpose the shape of the said tubular portion and/or seats or reliefs located on that tubular portion, suitable for cooperating with complementary elements of the connector of the external system.

During production, the support element 2g of the connection element or fluidic connector 2, 2′ could also be configured as an intermediate wall, which in the subsequent assembly step is provided with one or more openings for the connecting conductors 7.

1. A connection device for measuring at least one physical quantity of a fluid, comprising:
a tubular connection element having a front connection portion and a rear portion the tubular connection element defining a through cavity having a proximal opening and a distal opening opposite to each other, which are in the rear portion and in the front connection portion of the tubular connection element, respectively,
a detection unit configured for detecting at least one physical quantity characteristic of the fluid,
wherein the front connection portion of the tubular connection element is configured for connection, at least at or near the distal opening of the through cavity, to a fluidic system,
wherein the detection unit is mounted at the front connection portion of the tubular connection element, or closer to the distal opening than the proximal opening, and that the through cavity is closed at the front connection portion, or closer to the distal opening than the proximal opening (O₁).

2. The connection device according to claim 1, wherein the detection unit is mounted so as to close or contribute to the closure of the through cavity.

3. The connection device according to claim 1, wherein the detection unit is mounted inside the through cavity, in a recessed position with respect to the distal opening of the through cavity.

4. The connection device according to claim 3, wherein, between the detection unit and the distal opening of the through cavity, a medium is set in the through cavity configured for protecting the detection unit and for transferring the at least one physical quantity from the fluid to the detection unit (5).

5. The connection device according to claim 1, wherein the tubular connection element has, at the front connection portion thereof and inside the through cavity, at least one intermediate support element, configured for supporting the detection unit.

6. The connection device according to claim 1, wherein the detection unit comprises at least one element sensitive to the at least one physical quantity, and a substrate of the at least one element sensitive to the at least one physical quantity.

7. The connection device according to claim 6, wherein the tubular connection element has, at the front connection portion thereof and inside the through cavity, at least one intermediate support element, and wherein the substrate of the at least one element sensitive to the at least one physical quantity is sealingly constrained on the at least one intermediate support element.

8. The connection device according to claim 1, comprising a control circuit connected in signal communication with the detection unit, the control circuit extending within the through cavity, between the control circuit and the detection unit there extending first electrical conductors inside the through cavity.

9. The connection device according to claim 1, comprising an electrical connector or a multicore cable configured for electrical connection of the connection device to an external system.

10. The connection device according to claim 1, comprising a control circuit which includes a circuit support bearing:
circuit components configured for processing an electrical signal that can be acquired by means of the detection unit,
first terminals configured for connecting first electrical conductors for connection to the detection unit, and
second terminals of an electrical connector, or configured for connecting an electrical connector, or configured for connecting a multicore cable.

11. The connection device according to claim 9, wherein the tubular connection element has, at least at the rear portion thereof and inside the through cavity, at least one of a positioning guide of the circuit support and an abutment surface defining a position of maximum insertion of the circuit support into the through cavity.

12. The connection device according to claim 8, wherein a mass of electrically insulating fixing material is placed inside the through cavity, configured to secure in place the control circuit and/or at least part of an electrical connector and/or an end portion of a multicore cable for electrical connection of the connection device to an external system.

13. The connection device according to claim 1, where the detection unit comprises a pressure sensor.

14. The connection device according to claim 13, wherein the pressure sensor is a pressure sensor of a relative type defining an internal cavity, the internal cavity being connected in fluid communication to an ambient pressure via a tube.

15. The connection device according to claim 1, wherein the tubular connection element is configured at least in part as a Luer-type connector.

16. The connection device according to claim 1, comprising an annular fixing element, which surrounds a part of the tubular connection element in a region thereof between the rear portion and the front connection portion, the annular fixing element having an internal surface provided with fixing means, wherein:
the annular fixing element is formed in a single piece with the tubular connection element, or else
the annular fixing element is configured as a part separate from the tubular connection element and is mounted thereon in a rotatable manner.

17. A connection device for measuring at least one physical quantity of a fluid, comprising:
a tubular connection element having a front connection portion and a rear portion, the tubular connection element defining a through cavity having a proximal opening and a distal opening opposite to each other, which are in the rear portion and in the front connection portion of the tubular connection element, respectively,
a detection unit configured for detecting at least one physical quantity characteristic of the fluid,
a control circuit, connected in signal communication with the detection unit,
wherein the front connection portion of the tubular connection element is connectable, at least at or near the distal opening of the through cavity, to a fluidic system
and wherein:
the detection unit is mounted at the front connection portion of the tubular connection element, that is, closer to the distal opening than the proximal opening, and/or
the through cavity is closed at the front connection portion, that is, closer to the distal opening than the proximal opening, and/or
the control circuit extends at least partly inside the through cavity at the rear portion of the tubular connection element, and/or
in at least a part of the through cavity a mass of electrically insulating fixing material is arranged, configured for fixing in position the control circuit and/or at least part of an electrical connector and/or an end portion of a multicore cable for electrical connection of the connection device to an external system, and/or
the detection unit comprises a relative-type pressure sensor defining an internal cavity, the internal cavity being connected in fluid communication to an ambient pressure by means of a tube, which extends at least in part inside the through-cavity, between the detection unit and the rear portion of the tubular connection element.

18. The connection device according to claim 2, wherein the detection unit is mounted so as to close in a sealed way or to contribute to the sealed closure of the through cavity in a sealed way.

19. The connection device according to claim 8, wherein the control circuit extends within the through cavity in a position spaced apart with respect to the detection unit.

20. The connection device according to claim 14, wherein the tube extends at least partially inside the through cavity, or between the detection unit and the rear portion of the tubular connection element.
